(19) United States
(12) Patent Application Publication
(10) Pub. No.: US 2002/0081565 A1
(43) Pub. Date: Jun. 27, 2002

(54) Process for producing freeze dried competent cells and use thereof in cloning

(75) Inventors: Efrat Barnea, Jerusalem (IL); Yael Asscher, Mevaseret Zion (IL); Castro Wattad, Jatt Village (IL)

Correspondence Address: SENNIGER POWERS LEAVITT AND ROEDEL, ONE METROPOLITAN SQUARE, 16TH FLOOR, ST LOUIS, MO, 63102, US

(73) Assignee: SIGMA-ALDRICH CO.

(21) Appl. No.: 09/973,444

(22) Filed: Oct. 9, 2001

Related U.S. Application Data
(60) Provisional application No. 60/244,359, filed on Oct. 30, 2000.

(51) Int. Cl. A01N 1/02; C12N 1/20

(52) U.S. Cl. 435/2; 435/252.1

(57) ABSTRACT

A process for producing lyophilized competent cells wherein competent cells are that can be stored or shipped as freeze-dried cells at temperatures between 0° C. and 8° C. and remain suitable for cloning genes or DNA fragments. The process includes culturing cells, rending the cells competent, and lyophilizing the cells. Once lyophilized, the cells can be stored or shipped as freeze-dried cells. The lyophilized cells are prepared for transformation protocols by being re-hydrated in a solution of dimethyl sulfoxide. Once re-hydrated, the transformation efficiency of the competent cells is at least 5×10⁵ transformations per microgram of DNA.

### BACKGROUND OF INVENTION

1. Field of the Invention

The present invention generally relates to a process for producing competent cells that are capable of being shipped and stored at temperatures at or above about 0° C.

2. Description of Related Art

The advancement of biotechnology and genetic engineering activities in recent years has resulted in rapid growth in utilizing cell cultures of host cells to cost effectively clone genes or produce biochemical molecules in useful quantities. Generally, this is done using recombinant DNA technology in which a gene or DNA fragment is isolated and inserted into a host cell as exogenous DNA. This is often done by inserting the isolated DNA sequence into a plasmid DNA molecule thereby forming a recombinant DNA molecule and inducing a bacterial cell to take up the recombinant DNA molecule. The process of inserting DNA into plasmids is described in U.S. Pat. No. 4,237,224 (Cohen and Boyer, 1980). Once inside the cell, the proteins encoded by the DNA may be produced in the cell through expression of the gene or fragment in the host cell. As the host cells reproduce, the plasmid (exogenous DNA) is also replicated, thereby increasing the amount of the gene or DNA fragment.

Essential to this process are host cells which have the ability to take up exogenous DNA. The process in which a host cell, such as bacterial, yeast, or plant cell, takes up the exogenous DNA and incorporates it into the genome of the cell is known as transformation. A cell that is able to undergo transformation is called a competent cell. Competent cells may be lower eukaryotic cells, such as a yeast cells, or prokaryotic cells, such as a bacterial cells, which are capable of transformation. Escherichia coli, a gram-negative bacteria, is often used as a host cell in recombinant technology.

A number of procedures exist for the preparation of competent E. coli cells and the introduction of the exogenous DNA into the host cell. For example, Mandel and High (1970, Journal of Molecular Biology 53:159) describe a procedure whereby E. coli cells are infected with bacteriophage DNA with in the presence of 50 mM Ca⁺⁺ at 0° C., followed by a brief heat pulse at 37° C. to 42° C. This method has been extended to the uptake of chromosomal DNA (Cosloy and Oishi, 1973, Proceedings of the National Academy of Science 70:84) and plasmid DNA (Cohen et al., 1972, PNAS 69:2110). A summary of the factors influencing the efficiency of transformation is given in Hanahan (1983, JMB 166:557). These factors include the addition of other cations such as Mg, Mn, or Rb to the Ca-treated cells, as well as the prolonged incubation of the cells in CaCl₂.

The transformation efficiency of a given cell line is the number of cells that are transformed per amount of DNA subjected to transformation process. The efficiency of transformation of E. coli cells is substantially enhanced by the method described by Hanahan (1983, JMB 166:557), hereinafter referred to as “Hanahan (1983).” Hanahan (1983) described the growth of E. coli at 37° C. in the presence of 20 mM Mg. Plasmid DNA was added to the cells and incubated at 0° C. in the presence of Mn, Ca, Rb or K, dimethylsulfoxide (DMSO), dithiothreitol (DTT) and hexamine cobalt chloride. The E. coli strains prepared by the Hanahan (1983) method have transformation efficiencies of 1×10⁸ to 5×10⁸ transformants/μg plasmid DNA.

A freezing method of competent cells for long-term storage is described in Hanahan (1983) wherein competent cells were frozen at −70° C. and stored for several months without significant loss of transformation efficiency. Generally, frozen competent cells have transformation efficiencies of about 1×10⁶ to 1×10⁸ transformants/μg plasmid DNA. In this method, transformed cells were grown in SOB medium, chilled on ice, made competent, flash-frozen in solid CO₂/EtOH, and stored at −70° C. This method permits long-term storage of competent cell stock where acceptable transformation efficiencies were obtained wherein the competent cells were frozen, thawed once, and used in transformation procedures. If the cells were frozen, thawed, and re-frozen, however, a reduction in the transformation efficiency of the competent cells was observed.

Jessee et al., U.S. Pat. No. 4,981,797 describe a process for producing competent cells which are grown at 18° C. to 32° C., frozen at −70° C., thawed, and re-frozen at −70° C. Greener, U.S. Pat. Nos. 5,512,468 and 5,707,841 describe gram negative bacteria, such as E. coli, that were genetically modified by the addition of a genetic construction for the expression of a carbohydrate degrading enzyme so as to exhibit increased transformation efficiency. Jessee et al. and Greener, however, describe freezing the cells at −70° C. according to the Hanahan,(1983) method for long-term storage.

Thus, competent cells produced using procedures developed to date, as well as commercially available competent cells (e.g. E. coli), have typically been shipped and stored at −70° C. to preserve their ability to be used over prolonged periods of time. These storage temperature requirements increase the cost of shipping and storing competent cells. In addition, the risk of losing competent cell stock is ever present in the event of a refrigeration failure during storage or when shipping delays or packaging failures occur, resulting in competent cell storage and shipping temperatures above −70° C.

Industry would therefore greatly benefit from a procedure for producing competent cells and the cells produced from that procedure that do not require long-term storage or shipping temperatures of −70° C.

### SUMMARY OF THE INVENTION

Accordingly, it is an object of the present invention to provide a process for producing competent cells that are grown, rendered competent, and lyophilized. The competent cells may be stored or shipped at temperatures at or above about 0° C. and remain suitable for cloning genes or DNA fragments.

A further object of the present invention are lyophilized competent cells produced by a process wherein the resultant cells may be stored and shipped at temperatures at or above about 0° C. and remain suitable for cloning genes or DNA fragments.

Briefly, therefore, the present invention is directed to a process in which competent cells are produced by growing cells in a growth conducive medium, rendering the cells competent, and lyophilizing the competent cells. Once lyophilized, the competent cells can be stored at temperatures between about 0° C. and about 8° C. and remain suitable for cloning genes or DNA fragments upon re-hydration.

In another aspect, the invention is directed to competent cells produced by growing the cells in a growth conducive medium, rendering the cells competent, and lyophilizing the competent cells. Once lyophilized, the competent cells can be stored at temperatures between about 0° C. and about 8° C. and remain suitable for cloning genes or DNA fragments upon re-hydration.

Other features of the present invention will be in part apparent to those skilled in the art and in part pointed out in the detailed description provided below.

### DETAILED DESCRIPTION OF THE PREFERRED EMBODIMENTS

The present invention relates to competent host cells and a process of producing competent host cells that are able to be stored or shipped at temperatures at or above about 0° C. Particularly, the invention relates to a process wherein cells are cultured, the cultured cells are rendered competent, and the competent cells are lyophilized to dryness in lyophilization vials.

It has been discovered that cells rendered completely or partially competent may be produced in a process wherein the competent cells are freeze-dried. The resulting freeze-dried cells may be stored at temperatures between about 0° C. to about 8°C. The cells may then be rehydrated and used in transformation processes without significant decline of transformation efficiency that will prevent utilization of the cells in regular transformation experiments.

The invention, in its preferred form, is a process for producing freeze-dried competent cells that may be stored for extended periods of time or shipped as a dry powder at temperatures between about 0° C. to about 8° C., more preferably at temperatures between about 0° C. to about 4° C., most preferably shipping on wet ice.

A successful gene or DNA fragment cloning procedure begins by first selecting and culturing a desired line of eukaryotic cells or prokaryotic cells. The cells can be lower eukaryotic cells, such as a yeast cells, or prokaryotic cells, such as a bacterial cells. Within these broad groups of cells, additional divisions exist, all of which are potential candidates for competent cells. For example, within the bacterial cells are gram positive and gram negative bacteria. While gram positive bacteria interact with double-stranded DNA, they transfer only one strand into the cell. Gram negative bacteria, however, both interact with and transfer double-stranded DNA into the cells. Within the group of gram negative bacteria exist many genera of bacteria that can be rendered competent. For example, bacteria of the Genera Agrobacterium, Escherichia, Klebsiella, Proteus, Pseudomonas, Rhizobium, Salmonella, and Shigella, among others, can be rendered competent and used in cell transformation procedures. These genera may be even further divided into acceptable strains of cell lines that may have unique beneficial qualities. For example, Escherichia coli, one of the most common cells used in molecular biology procedures, has several cell lines that can produce desirable competent cells. A non-exhaustive example of these lines include RR1, HB101, JM101, JM109, DH5α, DH1, LE392, BL21 among others. Thus, while often only a few cell lines may be commonly used in recombinant protocols based on supply, cost, degree of knowledge of a cell line, or other reason the present invention can be used to produce lyophilized competent cells cultured from a multitude of different cell lines.

Once the desired cell line is selected, a culture of the cell line is grown. This may be performed by streaking a drop of the selected cell line stock on a favorable growth media for the selected cell line and incubating at temperatures favorable for growth. The culturing and growth of individual cell lines are well known in the art. Once cultured, a single colony from the growth media may be selected to produce a starter culture of cells for the selected cell line.

A starter culture of cells may be grown in a medium to produce large quantities of cells from the selected cell line. This may be generated by incubating cells from the selected cell line, preferably from a single colony of the cell line, in a growth conducive medium at a favorable temperature for a period of time. A growth conducive medium may include a medium such as a SOC solution (described in Example 1) or other medium that promotes the growth of the selected cell line. The incubation period may be for several hours, preferably for about 12 to 18 hours, or overnight. During the incubation period, the medium is maintained at a temperature that is favorable to the growth of the selected cell line. For many of the cell lines this is between about 28° C. to 40° C., preferably about 37° C. The growth may be further promoted by subjecting the medium to mixing or agitation, preferably constant agitation, more preferably constant agitation at about 180 to 250 rpm.

When the starter culture contains a desired concentration of cells, it may be diluted and the cells further grown in growth conducive media. The starter solution may be diluted to about 1:50 to about 1:100 with the incubation media. The starter culture or diluted starter culture may be used to inoculate a freshly prepared SOB solution or other growth conducive media at a favorable temperature range for the selected cell line. For example, E. coli for obtaining competent cells has a favorable growth temperature between about 28° C. to 40° C., preferably between about 28° C. to about 32° C., more preferably at about 30° C.

The cells in the cell culture solution may be cultured at a favorable growth temperature on a rotary shaker for several hours. Preferably, the culture is incubated until it reaches a desired cell concentration. Cell concentrations may be measured by the optical density of the culture. For example, a culture of E. coli cells may be preferably incubated with constant agitation on a shaker at about 180 rpm to about 250 rpm at a temperature of about 30° C. The cells may be collected at a desired cell concentration, preferably when the optical density of the cells measured at 600 nm (OD₆₀₀) reaches 0.4 to 1.0, more preferably at an OD₆₀₀ of 0.45 to 0.6.

Cells may be rendered competent by using a cell competency protocol such as the Hanahan (1983) method, a modification of the Hanahan (1983) method, or other available protocol suitable for the production of competent cells. See Sambrook, J. et al., Molecular Cloning: A Laboratory Manual. 2^{nd} Edition, Cold Spring Laboratory, Cold spring Harbor, N.Y., 1989.

In one method of producing competent cells, a modification of the Hanahan (1983) method, cells from the cell culture may be rendered competent by first cooling the cell culture, preferably to temperatures of about 2° C. to about 4° C. by immersing the cell culture in an ice water bath for about 15 to 30 minutes. Aliquots of the cooled culture (e.g. about 50 mL to 1000 mL) may be transferred to sterile conic test tubes and centrifuged to concentrate the cells. Preferably, the test tube chamber of the centrifuge is pre-chilled to about 4° C. and the cells are centrifuged for about 10 minutes at speeds of about 1800× g to about 2,400× g.

The centrifuged cells are rendered competent by subjecting them to a competency buffer. This may be done by mixing or re-suspending the centrifuged cells in the competency buffer. The competency buffer is a chemical, chemical mixture, and/or solution that, when mixed with cells, makes the cells competent (e.g. CB-I as described in Examples 1-5 below).

In the modified Hanahan (1983) method, the supernatant in the centrifuged test tubes is carefully removed and discarded and CB-I buffer, preferably about 2 to 3 mL, is added to each of the test tubes. The cells may be re-suspended in the CB-I buffer by pulsing the cells and buffer in the test tube with a vortex. Preferably, the test tubes are pulsed about five times for about one second each on a full speed vortex. To ensure complete re-suspension of the cells, glass beads may be added to the test tubes and another vortex pulse to several pulses may be performed, depending on the amount of cell pellet. Additional competency buffer may be added to the re-suspended cells in the test tubes (e.g. about 10 to 17 mL of CB-I).

The cells, having been made competent by being mixed with the competency buffer, may be further subjected to an ice incubation process, be frozen and stored, be frozen and lyophilized without storing, or the competent cells may be immediately used in a transformation protocol. The re-suspended cells may be frozen by being placed directly in containers, such as lyophilization vials, and subjected to freezing temperatures. Preferably, the re-suspended cells may be placed in lyophilization vials and placed in contact with liquid nitrogen (e.g. set in a tray containing liquid nitrogen) wherein the re-suspended cells are immediately frozen (hereinafter referred to as “snap-freezing” or “snap-frozen”).

In the ice bath incubation step, the contents of tubes may be combined (e.g. every two test tubes containing re-suspended cells may be combined in a single test tube). Test tubes containing the re-suspended cells may be gently shaken in an ice bath for a period of time that is favorable for the selected cell line. For example, the DH5α cell line is preferably shaken in an ice bath for about 30 to about 60 minutes. If the cells were not combined prior to the ice bath incubation, they may be combined following the ice bath incubation.

To improve efficiency, the cells may be centrifuged, resuspended, and incubated in an ice bath several times. Preferably, the cells are centrifuged for about 10 minutes at about 1,800× g to 2,400× g at a temperature of about 4° C. The supernatant of the centrifuged tubes is carefully discarded and the cells are preferably re-suspended in about 5 mL of CB-I by pulsing the solution in the test tube about five times for about one second each on a full speed vortex with or without glass beads. The test tubes containing the re-suspended cells may be gently shaken in an ice bath for a period of time (e.g. about 15 to about 60 minutes). The process of adding glass beads, vortexing, and shaking in an ice bath may be repeated. At the end of the ice incubation step, aliquots of the re-suspended cells may be frozen. Preferably, aliquots of the re-suspended cells are placed in lyophilization vials with loose stoppers (e.g. about 0.1 to 0.5 mL per vial, such as a 0.5 mL, 2 mL, or 3.5 mL size vial) and snap-frozen as previously described.

The frozen competent cells may be stored at about −70° C. to about −80° C. or directly transferred to a pre-chilled lyophilizer. Frozen competent cells may be stored for one month to a few months at −70° C. without observing a decrease in transformation efficiency.

The frozen competent cells, whether previously subjected to ice bath incubation and frozen, frozen and stored, or recently frozen, may be freeze-dried by being placed in a lyophilizer. Preferably, the frozen competent cells are placed in a lyophilizer and lyophilized in the presence of a stabilizer such as galactose, glucose, maltose, raffinose, lactose, inositol, ectoine, and proline, preferably TB-Z, more preferably CB-I with sucrose, trehalose, or mixture thereof. At the time the frozen competent cells are placed in the lyophilizer, the lyophilization chamber that holds the cells is preferably prechilled to about −40° C. to −20° C., more preferably pre-chilled to about −30° C. The cells are preferably freeze-dried over several hours (e.g. about 20 to about 30 hours).

The lyophilized cells may be shipped to a remote location or stored as freeze-dried cells at about −80° C. to about 8° C., preferably about −20°0 C. to about 8° C., more preferably about 0° C. to about 8° C., most preferably about 0° C. to about 4° C. The freeze dried cells are most preferably shipped to remote locations on wet ice at a temperature of about 0° C. to about 4° C. The freeze-dried cells have the appearance of powder.

The lyophilized cells may be stored for approximately two years without significantly diminishing their competency characteristics. Additionally, the lyophilized cells may be stored for approximately 2 years at about −80° C. to about −20° C. without significantly diminishing their competency characteristics.

Before the freeze-dried cells may be utilized in transformation processes, they must first be rehydrated. This may be performed by mixing the freeze-dried cells with an aqueous solution and allowing the cells to absorb the solution. Preferably, the lyophilized competent cells are prepared for transformation by being re-suspended in lyophilization vials containing a re-hydration buffer for about 10 minutes. A preferred re-hydration buffer is a solution containing DMSO (e.g. approximately about 3.5% to about 7.0%) in ice water, more preferably, a solution containing DMSO and mercaptoethanol (e.g. about 0.014 M). A preferred re-hydration buffer is described in Examples 1-5. Following re-hydration, aliquots of the solution of re-hydrated cells (e.g. approximately 100 μl) may be dispensed into containers such as microfuge tubes and utilized as recipients of recombinant DNA for the purpose of cloning genes or DNA fragments. If lyophilized in plastic 100 μl vials, rehydration followed by transformation could be performed in the vial itself.

The re-hydrated cells may be subjected to transformation methods used in the art for competent cells that have not been subjected to lyophilization.

In a modification of the Hanahan (1983) method for transforming competent cells, transformation may be performed by adding the recombinant DNA (e.g. plasmids containing an inserted gene or DNA fragment) to containers holding the re-hydrated competent cells. The recombinant DNA and competent cells may be mixed by gentle finger tapping on the containers. Tubes containing the recombinant DNA and re-hydrated competent cells may be incubated on ice for approximately 30 minutes. Following the ice bath, the cells are subjected to heat shock in which the microfuge tubes are transferred to a water bath at a temperature of about 42° C., incubated for about 30 seconds, then transferred back into ice for about 2 minutes. Heat shock temperatures and time may be modified from about 37° C. to about 44° C. where the time duration from about 5 minutes for temperatures of approximately 37° C. to about 20 seconds for temperatures of approximately 44° C. SOC medium (e.g. about 200 μl to about 900 μl) are added to each of the tubes and the tubes are placed in a shaker and shaken for one hour at about 37° C.

The bacteria transformed with 10 ng DNA in each one of the tubes may be diluted to between about 1:20 to about 1:100 and plated on a growth conducive medium at a favorable growth temperature for the selected cell line (e.g. LB-AMP plate at 37° C. for bacteria transformed with ampicillin selection marker).

The lyophilized competent cells produced by the process of the present invention, when re-hydrated, provide transformation efficiencies suitable for cloning genes or DNA fragments of approximately 5×10⁵ transformations or more per microgram DNA.

As lyophilized cells can be stored and shipped at temperatures at or above about 0° C., the present invention results in reducing costs to the biotechnology industry that heretofore were required to store and ship competent cells at about −70° C. The invention also reduces the risk of losing competent cell stock in the event that either the storage or shipping refrigeration fails.

The invention is further illustrated by means of the following examples.

### EXAMPLE 1

### Lyophilized Cell Transformation Using Sucrose

Buffers and growth medium:

| | |
|---|---|
| CB-I (Competency buffer-I) | |
| Potassium acetate, pH 6.8, | 10 mM |
| CaCl₂ | 10 mM |
| MnCl₂ | 60 mM |
| KCl | 100 mM |
| Hexamine cobalt Chloride | 3 mM |
| Sucrose | 100 mM |
| Re-hydration Buffer | |
| 7% DMSO in ice-cold water | |
| 0.014 M Mercaptoethanol | |
| SOB | |
| Bacto tryptone | 20 g/liter |
| Yeast extract | 5 g/liter |
| NaCl | 0.5 g/liter |
| KCl | 2.5 mM |
| MgCl₂ | 10 mM |
| SOC | |
| Bacto tryptone | 20 g/liter |
| Yeast extract | 5 g/liter |
| NaCl | 0.5 g/liter |
| KCl | 2.5 mM |
| MgCl₂ | 10 mM |
| Glucose | 20 mM glucose |
| LB Plate Solution | |
| Tryptone | 10 g/liter |
| Yeast extract | 5 g/liter |
| NaCl | 5 g/liter to 10 g/liter |
| Agar | 15 g/liter |

Cell Culturing:

A drop of glycerol stock of DH5α E. coli was streaked on a LB plate and incubated overnight at 37° C. for approximately 12-18 hours. A single colony from the mentioned plate was used to produce a starter of E. coli. This starter was used at a dilution of 1:100 to inoculate freshly prepared SOB or other growth conducive media at a temperature of 30° C. or 37° C. Cells were cultured at approximately 30° C. on rotary shaker at a speed of about 180 to about 250 rpm/min for 3 to 10 hours. The optical density (OD₆₀₀) of the cells was measured at 600 nm. The cells were collected upon reaching an OD₆₀₀ of 0.4 to 1.0, more preferably an OD₆₀₀ of 0.45 to 0.6.

Competent Cells Preparation:

Upon reaching the desired optical density, cells were cooled to 2° C. to 4° C., and rendered competent by using a modified Hanahan procedure, as outlined below.

Method:

A single colony of E. coli DH5α bacteria was incubated in 5 mL SOC medium (Catalog no. S-1797, Sigma, St. Louis, Mo.) and cultured at about 37° C. with constant agitation at about 180 to about 250 rpm. After being cultured overnight for approximately 12 to 18 hours, 2 mL of the culture was added to approximately 200 mL SOB medium in a 2 liter flask. The culture was incubated with constant agitation at 180 to 250 rpm for approximately 5 hours at about 30° C. until an optical density (OD₆₀₀) of 0.45 measured at 600 nm was reached. The culture was immersed for 20 minutes in an ice-water bath.

The culture was transferred to four 50 mL conic sterile test tubes and sedimented by centrifugation for 10 minutes at 2420 g using a Sorvall SS-34 rotor (Kendro Laboratory Products, Newton, Conn.) pre-chilled to approximately 4° C.

The supernatant was discarded and 3 mL CB-I buffer was added to each of the test tubes at a temperature of about 4° C. Cells were then re-suspended by five 1 second pulses of full speed vortex (Vortex Genie 2, Scientific Industries, Inc., Bohemia, N.Y.). To ensure complete re-suspension, approximately 7 grams (about 5 mL in volume) of glass beads were added to each tube and another vortex pulse was performed. Then, 12 mL of CB-I was added to each of the 4 tubes and re-suspended bacteria were gently shaken on ice for 5 minutes. The re-suspended cells were collected from the 4 tubes into two 50 mL test tubes and precipitated again by centrifugation as described above (centrifuged at 4° C. for 10 minutes at 2420 g).

The supernatant was discarded. The process of re-suspension in 3 mL CB-I, followed by the addition of glass beads, 12 mL CB-I and shaking on ice for 15 minutes was repeated. Cells in suspension were collected into a single tube and centrifuged again as described above.

The supernatant was discarded, glass beads and 10 mL CB-I buffer was added to the tubes, and the cells were re-suspended with five 1 second full speed vortex impulses. The cells were then collected into a new test tube.

Of the total volume obtained, 2.5 mL were transferred to a new test tube and incubated on ice for 15 minutes in the presence of 88 μl dimethyl sulfoxide (DMSO) to serve as a control for transformation without lyophilization. After 15 minutes, another 88 μl of DMSO was added. The control cells were divided into 100 μl portions, snap-frozen in liquid nitrogen, and stored at −70° C.

The remaining 7.5 mL were dispensed into 0.5 mL aliquots and snap-frozen in 3.5 mL clear glass lyophilization vials. These were stored at −70° C. for approximately 30 minutes up to 110 hours followed by lyophilization. A lyophilizer (Unitop 200, The VirTis Company, Inc., Gardiner, N.Y.) was cooled to −30° C. The lyophilization tubes containing the snap-frozen competent cells were placed in the lyophilizer and the lyophilization process was initiated. The lyophilization procedure lasted approximately 24 hours with an initial temperature of −30° C. that gradually increased in temperature over the lyophilization period to an ending temperature of 25° C. according to the lyophilization program described in Table 1.

TABLE 1
The Lyophilization Program

| Time | Temperature | Lyophilization Program Description |
|---|---|---|
| hour 0 | −30° C. | Climb to and remain at −25° C. within 1 |
| | | hour |
| hour 1 | −25° C. | Climb to and remain at −20° C. within 1 |
| | | hour |
| hour 2 | −20° C. | Climb to and remain at −15° C. within 1 |
| | | hour |
| hour 3 | −15° C. | Climb to and remain at −10° C. within 1 |
| | | hour |
| hour 4 | −10° C. | Climb to and remain at −5° C. within 1 hour |
| hour 5 | −5° C. | Climb to and remain at −0° C. within 1 hour |
| hours 6-16 | 0° C. | Remain at 0° C. for 10 hours. After 10 |
| | | hours, climb to and remain at 5° C. within |
| | | 30 minutes. |
| hour 16.5 | 5° C. | Climb to and remain at 10° C. within 30 |
| | | minutes. |
| hour 17 | 10° C. | Climb to and remain at 15° C. within 30 |
| hour 17.5 | 15° C. | Climb to and remain at 20° C. within 30 |
| hour 18 | 20° C. | Climb to and remain at 25° C. within 30 |
| | | minutes. |
| hours 18.5 | 25° C. | Remain at 25° C. for 6 hours. |
| to 24.5 | | |

Transformation was performed parallel in the control bacteria and lyophilized bacteria. The control bacteria were thawed on ice for 10 minutes. Vials containing lyophilized bacteria were re-suspended in re-hydration buffer (500 μl containing DMSO) and incubated on ice for the same period of time (10 minutes), followed by dispensing 100 μl aliquots into microfuge tubes.

Transformation was performed using a commercial pUC19 plasmid (Catalog no. D-3404, Sigma, St. Louis, Mo.). The plasmid DNA (10 ng) was added into each of the control competent cells tubes and re-hydrated lyophilized competent bacteria microfuge tubes. The plasmid and competent cells were mixed by gentle finger tapping on each tube. The tubes were then incubated on ice for 30 minutes. The cells were then subjected to heat shock in which the tubes were transferred to a 42° C. water bath and incubated for 30 seconds, then transferred back into the ice bucket for 2 minutes. SOC medium (200 μl to 900 μl) was added to each of the tubes and the samples were placed in a shaker or shaker incubator and shaken for 1 hour at 37° C.

The transformed bacteria in each of the tubes were diluted and plated on LB-AMP plate and grown at 37° C. After approximately 16 hours, the colonies were counted. The transformation results are presented in Table 2.

TABLE 2
Example 1 Transformation Results

| Yield | |
|---|---|
| (colonies/ | |
| μg DNA) | Treatment |
| approx. | Lyophilized bacteria preserved with sucrose (CB-I |
| 1 × 10⁶ | sucrose) |
| 1 × 10⁸ | Control-no lyophilization CB-I sucrose |

### EXAMPLE 2

### Lyophilized Cell Transformation Using Trehalose

Buffers and growth medium:

| | |
|---|---|
| CD-I (Competency buffer-I) | |
| Potassium acetate, pH 6.8, | 10 mM |
| CaCl₂ | 10 mM |
| MnCl₂ | 60 mM |
| KCl | 100 mM |
| Hexamine cobalt Chloride | 3 mM |
| Trehalose | 100 mM |
| Re-hydration Buffer | |
| 7% DMSO in ice-cold water | |
| 0.014 M Mercaptoethanol | |
| SOB | |
| Bacto tryptone | 20 g/liter |
| Yeast extract | 5 g/liter |
| NaCl | 0.5 g/liter |
| KCl | 2.5 mM |
| MgCl₂ | 10 mM |
| SOC | |
| Bacto tryptone | 20 g/liter |
| Yeast extract | 5 g/liter |
| NaCl | 0.5 g/liter |
| KCl | 2.5 mM |
| MgCl₂ | 10 mM |
| Glucose | 20 mM glucose |
| LB Plate Solution | |
| Tryptone | 10 g/liter |
| Yeast extract | 5 g/liter |
| NaCl | 10 g/liter |
| Agar | 15 g/liter |

Method:

The cell culturing, competent cell preparation, and method is the same as identified in Example 1 above, except that the CB-I buffer contains trehalose instead of sucrose as a preservative. The transformation results are presented in Table 3.

TABLE 3
Example 2 Transformation Results

| Yield | |
|---|---|
| (colonies/ | |
| μg DNA) | Treatment |
| 5 × 10⁵ | Lyophilized bacteria preserved with trehalose (CB-I |
| | trehalose) |
| 6 × 10⁷ | Control-no lyophilization CB-I trehalose |

### EXAMPLE 3

### Lyophilized Cell Transformation Using Sucrose or Sucrose and Trehalose

| | |
|---|---|
| CB-I (Competency buffer-I) | |
| Potassium acetate, pH 6.8, | 10 mM |
| CaCl₂ | 10 mM |
| MnCl₂ | 60 mM |
| KCl | 100 mM |
| Hexamine cobalt Chloride | 3 mM |
| Sucrose | 100 mM |
| CB-I (200) | |
| Potassium acetate, pH 6.8 | 10 mM |
| CaCl₂ | 10 mM |
| MnCl₂ | 60 mM |
| KCl | 100 mM |
| Hexamine cobalt chloride | 3 mM |
| Sucrose | 200 mM |
| CB-T (100 + 100) | |
| Potassium acetate, pH 6.8 | 10 mM |
| CaCl₂ | 10 mM |
| MnCl₂ | 60 mM |
| KCl | 100 mM |
| Hexamine cobalt chloride | 3 mM |
| Sucrose | 100 mM |
| Trehalose | 100 mM |
| Re-hydration Buffer | |
| 7% DMSO in ice-cold water | |
| 0.014 M Mercaptoethanol | |
| SOB | |
| Bacto tryptone | 20 g/liter |
| Yeast extract | 5 g/liter |
| NaCl | 0.5 g/liter |
| KCl | 2.5 mM |
| MgCl₂ | 10 mM |

Method

A single colony of E. coli DH5α bacteria was incubated in two 5 mL SOC media and cultured overnight for approximately 15 hours at 37° C. with constant agitation at 250 rpm. The culture was diluted 1:100 in two 2-liter Erlenmeyer flasks containing 400 mL of SOB medium. The cultures were incubated with constant agitation at 250 rpm, for approximately 5 hours at 30° C. until reaching OD₆₀₀ of 0.53. The cultures were incubated for 20 minutes in an ice-water bucket.

The cultured cells were transferred to eighteen 50-mL conic sterile test tubes. All tubes were centrifuged for 10 minutes at 2,240 g using a fixed angle rotor (Sorvall SS-34) pre-chilled to approximately 4° C.

The supernatant was carefully discarded and the cells were re-suspended by five 1-second pulses of full speed vortex and one pulse in the presence of glass beads in 3 mL buffer as follows:

12 tubes were re-suspended in CB-I;

4 tubes were re-suspended in CB-I (200); and

2 tubes were re-suspended in CB-I (100+100).

12 mL of the appropriate buffer (e.g. CB-I if re-suspended in CB-I) was added to each of the tubes. The tubes were shaken on ice for 50 minutes. The contents of the shaking tubes were transferred to fresh test tubes with no glass beads. Every two tubes of each treatment were combined in one test tube.

The tubes were centrifuged. The supernatant was discarded and cells were re-suspended by five 1-second pulses of full speed vortex and one pulse in the presence of glass beads in 5 mL of the buffer used for the previous re-suspension. The suspended bacteria of each treatment were transferred to one fresh test tube with no glass beads.

One and a half milliliters from each tube was transferred to another test tube and incubated on ice for 15 minutes in the presence of 52.5 μl of DMSO. After 15 minutes, an additional 52.5 μl of DMSO was added and aliquots of 100 μl cells were dispensed into 1.5 mL microfuge tubes, snap-frozen in liquid nitrogen and stored at −70° C. Meanwhile, the rest of the suspended bacteria of the different treatments were snap-frozen in 0.5 and 0.1 mL aliquots in 3.5 mL clear lyophilization vials with a loose stopper.

The frozen cells were stored at −70° C. for 24 hours and then transferred to a pre-chilled lyophilizer. The cells were lyophilized according to the lyophilization program described in Table 1.

Several vials were taken for transformation. The rest of the vials were stored at −70° C., −20° C., 4° C., 25° C., 37° C. and 50° C. Transformation experiments were performed immediately after lyophilization and after different storage durations.

Transformation

The “control” bacteria which had not been lyophilized were defrosted on ice for 10 minutes, as the vials containing the lyophilized bacteria were re-suspended in Re-hydration Buffer (500 μl/vial) and incubated on ice for the same period of time (10 minutes). Following the incubation, the re-hydrated lyophilized bacteria were transferred to microfuge tubes at 100 μl aliquots.

Ten nanograms of a commercial PUC19 plasmid was added to the control and lyophilized bacteria samples (in the microfuge tubes), mixed by a gentle finger tapping on the tubes and incubated on ice for 30 minutes. The cells were then subjected to heat shock in which the tubes were transferred to 42° C. water bath for 30 seconds and transferred back to the ice bucket for 2 minutes. 900 μl SOC medium was added to each one of the test tubes and samples were shaken for 1 hour at 37° C.

The transformed bacteria in each one of the tubes were diluted 1:20 and 1:100 (for the lyophilized cells) or 1:2000 and 1:10,000 (for the non-lyophilized cells) with SOC medium. Transformed bacteria, in 50 or 100 μl aliquots from each one of the dilutions, were plated on a LB-AMP plate and grown at 37° C. After approximately 16 hours, colonies were counted. The transformation results are presented in Tables 4 and 5.

TABLE 4
Example 3 Transformation results

| | | | Yield before | Yield after |
|---|---|---|---|---|
| | | Storage | lyophilization | lyophilization |
| | | Temp. | colonies/μg | colonies/μg |
| Day | Treatment | ° C. | plasmid | plasmid |
| Immediate | CB-I(200) | | 2.6 × 10E8 | 4 × 10E6 |
| Immediate | CB-I(100 + 100) | | 2.9 × 10E8 | 7.8 × 10E6 |
| 2 | CB-I(200) | −70 | 3.3 × 10E8 | 7.8 × 10E6 |
| 2 | CB-I(100 + 100) | −70 | 3.2 × 10E8 | 3.4 × 10E6 |
| 2 | CB-I(200) | −20 | | 3.7 × 10E6 |
| 2 | CB-I(100 + 100) | −20 | | 5.3 × 10E6 |
| 2 | CB-I(200) | 4 | | 3.6 × 10E6 |
| 2 | CB-I(100 + 100) | 4 | | 1.5 × 10E7 |
| 2 | CB-I(200) | 25 | | 3 × 10E4 |
| 2 | CB-I(100 + 100) | 25 | | 1 × 10E6 |
| 2 | CB-I(200) | 37 | — | — |
| 2 | CB-I(200) | 50 | — | — |
| 4 | CB-I(200) | −70 | 3.8 × 10E8 | 1 × 10E7 |
| 4 | CB-I(100 + 100) | −70 | 2.7 × 10E8 | 1.2 × 10E7 |
| 4 | CB-I(200) | −20 | | 2 × 10E6 |
| 4 | CB-I(100 + 100) | −20 | | 1.1 × 10E7 |
| 4 | CB-I(200) | 4 | | 1.2 × 10E6 |
| 4 | CB-I(100 + 100) | 4 | | 2 × 10E6 |
| 4 | CB-I(200) | 25 | | 3 × 10E4 |
| 4 | CB-I(100 + 100) | 25 | | 1 × 10E4 |
| 7 | CB-I(200) | −70 | 1.7 × 108 | 9.8 × 10E6 |
| 7 | CB-I(200) | −20 | | 2.1 × 10E6 |
| 7 | CB-I(200) | 4 | | 4.7 × 10E6 |
| 7 | CB-I(200) | 25 | | — |
| 14 | CB-I(200) | −70 | 1.4 × 10E8 | 4.3 × 10E6 |
| 14 | CB-I(200) | −20 | | 3 × 10E6 |
| 14 | CB-I(200) | 4 | | 1.7 × 10E6 |
| 14 | CB-I(200) | 25 | | — |

TABLE 5
Transformation of CB-I Preserved Lyophilized Cells

| | Storage | Yield before | Yield after |
|---|---|---|---|
| | Temp. | lyophilization | lyophilization |
| day | ° C. | Colonies/μg plasmid | Colonies/μg plasmid |
| Immediate | | 5.2 × 10E8 | 1.8 × 10E6 |
| Immediate | | 1.7 × 10E8 | 5.4 × 10E6* |
| 1 | −70 | 3 × 10E8 | 9.3 × 10E6 |
| 1 | −70 | 3 × 10E8 | 2.7 × 10E6 |
| 1 | −70 | 3 × 10E8 | 1.9 × 10E6* |
| 1 | −20 | 2.7 × 10E8 | 7.8 × 10E6 |
| 1 | −20 | 2.7 × 10E8 | 8.5 × 10E6* |
| 1 | 4 | | |
| 1 | 4 | | |
| 1 | 25 | | |
| 1 | 37 | — | — |
| 1 | 50 | — | — |
| 2 | −70 | 4.9 × 10E8 | 6.5 × 10E6 |
| 2 | −20 | | 5.9 × 10E6 |
| 2 | 4 | | 9.5 × 10E6 |
| 2 | 25 | | 1.3 × 10E5 |
| 3 | −70 | 1.7 × 10E8 | 9 × 10E6 |
| 3 | −70 | 1.7 × 10EB | 1 × 10E7* |
| 3 | −20 | 6.7 × 10E8 | 6 × 10E6 |
| 3 | −20 | 6.7 × 10E8 | 8.5 × 10E6* |
| 3 | 4 | | 8.4 × 10E6 |
| 3 | 4 | | 2.2 × 10E6* |
| 3 | 25 | | 5.4 × 10E5 |
| 4 | −70 | 3.9 × 10E8 | 1.1 × 10E7 |
| 4 | −70 | 3.9 × 10E8 | 1.2 × 10E7 |
| 4 | −20 | 1.9 × 10E8 | 6.1 × 10E6 |
| 4 | 4 | | 5.2 × 10E6 |
| 4 | 25 | | 1 × 10E4 |
| 4 | 25 | | 1.8 × 10E5 |
| 5 | −70 | | 7.6 × 10E6 |
| 5 | −20 | 2.9 × 10E8 | |
| 5 | 4 | | 1.2 × 10E6 |
| 6 | −70 | 7 × 10E8 | 1.6 × 10E7 |
| 6 | −70 | 7 × 10EB | 2.5 × 10E7* |
| 6 | −20 | 6 × 10E8 | 1.6 × 10E6 |
| 6 | −20 | 6 × 10E8 | 8.7 × 10E6* |
| 6 | 4 | | 7.6 × 10E6 |
| 6 | 4 | | 7.3 × 10E6* |
| 6 | 25 | | 5 × 10E4 |
| 7 | −70 | 3.4 × 10E8 | 6.6 × 10E6 |
| 7 | −20 | | 8.4 × 10E6 |
| 7 | 4 | | 5.7 × 10E6 |
| 14 | 70 | 3 × 10E8 | 5.2 × 10E6 |
| 14 | −20 | | 8 × 10E6 |
| 14 | 4 | | 3.2 × 10E6 |
| 21 | −70 | 1 × 10E8 | |
| 21 | 4 | | 6.4 × 10E6 |
| 28 | −70 | 1.4 × 10E8 | 4.3 × 10E6 |
| 28 | −20 | | 2.3 × 10E6 |
| 28 | 4 | | 1.2 × 10E6 |

*Results of the transformation of cells lyophilized at 0.1-mL aliquots.

### EXAMPLE 4

| | |
|---|---|
| CB-I (Competency Buffer-I) | |
| Potassium acetate, pH-6.8 | 10 mM |
| CaCl₂ | 10 mM |
| MnCl₂ | 60 mM |
| KCl | 100 mM |
| Hexamminecobalt Chloride | 3 mM |
| Sucrose | 100 mM |
| Re-hydration Buffer | |
| 7% DMSO in ice-cold water | |
| 0.014 M β-Mercaptoethanol | |
| SOB | |
| Bacto tryptone | 20 gr/liter |
| Yeast extract | 5 gr/liter |
| NaCl | 0.5 gr/liter |
| KCl | 2.5 mM |
| MgCl₂ | 10 mM |

Method

A single colony of E. coli DH5α bacteria was incubated in 5 mL of SOC medium (“starter”) and cultured overnight for approximately 15 hours at 37° C. with constant agitation at 250rpm. The culture was diluted 1:100 as two 2.5 mL aliquots of the culture were diluted in two 2-liter Erlenmeyer flasks containing 250 mL of SOB medium each. The culture was incubated with constant agitation at 250 rpm, for approximately 5 hours at 30° C. to approximately O.D.₆₀₀ 0.49. The bacteria culture was incubated for 20 minutes in an ice-water bucket.

The contents of the two Erlenmeyer flasks were collected together and transferred to ten 50 mL sterile conic test tubes. All tubes were centrifuged for 10 minutes at 2,240 g in a pre-cooled centrifuge. Four tubes were centrifuged in 5810 R Eppendorf centrifuge, using a swinging-out rotor (Treatment A). Six tubes were centrifuged in a Sorvall SS-34 fixed angle rotor (Treatment B). The supernatant was carefully discarded and the cells were re-suspended. The contents of the tubes for Treatments A and B were treated as follows:

Treatment A—The contents of two tubes were re-suspended in 3 mL of CB-I by five 1-second full speed vortex and a passage through a pipette. The other two tubes were re-suspended in 3 mL of CB-I by five 1-second full speed vortex, then 7 grams of glass beads were added and the bacteria was subjected to additional 1-second full speed vortex. The final re-suspension has been achieved by a passage through a pipette. Finalizing bacteria re-suspension, 12 mL of CB-I was added to each of the tubes and the contents of every two tubes mentioned above were combined in a fresh tube.

Treatment B—The contents of two tubes were re-suspended in 3 mL of CB-I and the contents of the other two tubes were re-suspended in 2.5 mL CB-I by five 1-second full speed vortex, addition of glass beads, and another 1-second full speed vortex. The contents of the additional two tubes were re-suspended in 3 mL of CB-I by five 1-second full-speed vortex and a passage through a pipette. Finalizing bacteria re-suspension, 12 mL of CB-I was added to the four tubes re-suspended in 3 mL CB-I and the contents of every two tubes of the same re-suspension treatment (containing or not containing glass beads) were combined.

All tubes except for one of the two re-suspended in 2.5 mL CB-I were gently shaken in an ice-water bucket for 50 minutes.

Half a milliliter of the bacteria in the tube not to be shaken was transferred to a fresh test tube and incubated on ice for 15 minutes in the presence of 17.5 μl of DMSO. After 15 minutes, an additional 17.5 μl of DMSO was added and 100 μl aliquots of cells were dispensed into 1.5 mL microfuge tubes, snap-frozen in liquid nitrogen, and stored at −70° C. Meanwhile, the 2 mL suspended bacteria remaining in the tube were snap-frozen in 0.5 mL aliquots in 3.5 mL clear lyophilization vials with loose stoppers.

Following 50 minutes of ice bath incubation, 0.5 mL of the bacteria in the tube containing 2.5 mL re-suspended bacteria was transferred to a fresh test tube and incubated on ice for 15 minutes in the presence of 17.5 μl of DMSO. After 15 minutes, an additional 17.5 μl of DMSO was added and 100 μl aliquots of cells were dispensed into 1.5 mL microfuge tubes, snap-frozen in liquid nitrogen, and stored at −70° C. The remaining 2 mL in the tube was snap-frozen in 0.5 mL aliquots in 3.5 mL clear lyophilization vials with loose stoppers. The rest of the tubes were centrifuged for 10 minutes at 2,240 g in a pre-cooled swinging bucket (treatment A) or fixed angle bucket (treatment B) centrifuge. Three milliliters of CB-I was added to each one of the tubes and the tubes were re-suspended in the same way the first re-suspension was performed. Two milliliters of CB-I buffer was added to each one of the tubes and the bacteria were gently mixed. One milliliter from each tube was transferred to a fresh test tube and incubated on ice for 15 minutes in the presence of 35 μl of DMSO. At the end of the incubation, 35 μl of DMSO was added to the tubes and gently mixed. The cells were dispensed in 100 μl aliquots into 1.5 mL microfuge tubes, snap-frozen in liquid nitrogen, and stored at −70° C.

Meanwhile, the suspended cells with no DMSO were snap-frozen in 0.5 mL aliquots in 3.5 mL clear lyophilization vials with loose stoppers. The frozen cells were stored at −70° C. for 48 hours and then transferred to a pre-chilled lyophilizer.

The lyophilization program outlined above in Example 1, Table 1, was used in the lyophilization process.

One vial of each experiment was taken for transformation. The rest of the vials were stored at −20° C. On the following day some of the stored vials were used for a second transformation.

Transformation

The “control” bacteria which were not lyophilized were defrosted on ice for 10 minutes while the vials containing the lyophilized bacteria were re-suspended in Re-hydration Buffer (500 μl/vial) and incubated on ice for the same period of time (10 minutes). Following the incubation, 100 μl aliquots of the re-hydrated lyophilized bacteria were transferred to microfuge tubes.

Ten nanograms of PUC19 plasmid was added to the control and lyophilized bacteria samples in the microfuge tubes, mixed by gentle finger tapping on the tubes, and incubated on ice for 30 minutes. The cells were then subjected to a heat shock. The tubes were transferred to a 42° C. water bath for 30 seconds and transferred back to the ice bucket for 2 minutes. 900 μl of SOC medium was added to each one of the test tubes and samples were shaken for 1 hour at 37° C.

The transformed bacteria in each one of the tubes was diluted 1:20 and 1:100 (for the lyophilized cells) or 1:200 and 1:2000 (for the non-lyophilized cells). Fifty microliters of transformed bacteria from each one of the dilutions were plated on a LB-AMP plate and grown at 37° C. Colonies were counted after approximately 16 hours.

TABLE 6
Example 4 Transformation Results

| | Yield before | Yield after |
|---|---|---|
| | lyophilization | lyophilization |
| Treatment | Colonies/μg plasmid | Colonies/μg plasmid |
| Lyophilized bacteria, | 7.6 10E7 | |
| treatment A − | 9 × 10E7 | 3 × 10E5 |
| glass beads | | 8 × 10E5 |
| Lyophilized bacteria | 8.8 × 10E7 | |
| treatment A + | 9 × 10E7 | 1.7 × 106 |
| glass beads | | 3.4 × 10E5 |
| Lyophilized bacteria, | 1.1 × 10E8 | |
| treatment B − | 1 × 10E8 | 4.3 × 10E6 |
| glass beads | | 3.7 × 10E6 |
| | | 1.6 × 10E6 |
| Lyophilized bacteria, | 1.5 × 10E8 | |
| treatment B + | 2.1 × 10E8 | 6.1 × 10E6 |
| glass beads | | 2.2 × 10E6 |
| | | 1.9 × 10E6 |
| Lyophilized bacteria, | 1.5 × 10E8 | |
| treatment B, dispensing | approx. 1 × 10E8 | 1.65 × E6 |
| after first centrifugation | 1 × 10E8 | 2 × 10E5 |
| | | 2.3 × 10E6 |
| Lyophilized bacteria, | 1.1 × 10E8 | 5 × 10E8 |
| treatment B, dispensing | 2.3 × 10E8 | <10E5 |
| after 50 minutes on ice, | 3 × 10E8 | 1 × 10E5 |
| no second centrifugation. | | |

### EXAMPLE 5

| CB-I (Competency Buffer-I) | | |
|---|---|---|
| Potassium acetate, pH-6.8 | 10 | mM |
| CaCl₂ | 10 | mM |
| MnCl₂ | 60 | mM |
| KCl | 100 | mM |
| Hexamminecobalt Chloride | 3 | mM |
| Sucrose | 100 | mM |
| Re-hydration Buffer | | |
| 7% DMSO in ice-cold water | | |
| 0.014 M β-Mercaptoethanol | | |
| SOB | | |
| Bacto tryptone | 20 | gr/liter |
| Yeast extract | 5 | gr/liter |
| NaCl | 0.5 | gr/liter |
| KCl | 2.5 | mM |
| MgCl₂ | 10 | mM |

Method

A single colony of E. coli DH5α bacteria was incubated in 5 mL SOC medium (“starter”) and cultured overnight for approximately 15 hours at 37° C. with constant agitation at 250 rpm. The culture was diluted 1:100 by combining two 2.5 mL aliquots of the culture in two 2-liter Erlenmeyer flasks containing 250 mL of SOB medium. The culture was incubated with constant agitation at 250 rpm for approximately 5 hours at 30° C. until reaching an O.D.₆₀₀ of approximately 0.49. The bacteria culture was incubated for 20 minutes in an ice-water bucket. The contents of the two Erlenmeyer flasks were collected together and transferred to six 50 mL conic sterile test tubes. All tubes were centrifuged for 10 minutes at 1,700 g in a pre-cooled, fixed angle rotor (Sorvall SS-34). The supernatant was carefully discarded and the cells were re-suspended as follows:

1. One tube was re-suspended in 3 mL of CB-I, two tubes in 2.5 mL CB-I and one tube in 5 mL CB-I, by five 1-second full speed vortex, addition of glass beads and another 1 second full speed vortex. The suspended bacteria was transferred to a fresh test tube (without the glass beads).

2. One tube was re-suspended in 3 mL of CB-I, by five 1-second full speed vortex and a passage through a pipette.

Finalizing bacteria re-suspension, 12 mL of CB-I was added to the two tubes re-suspended in 3 mL CB-I.

All tubes except for one out of the two re-suspended in 2.5 mL CB-I were gently shaken in an ice-water bucket for 50 minutes.

Half a milliliter of the bacteria in the tube not to be shaken was transferred to a fresh test tube and incubated on ice for 15 minutes in the presence of 17.5 μl of DMSO. After 15 minutes, an additional 17.5 μl of DMSO was added and aliquots of 100 μl cells were dispensed into 1.5 mL microfuge tubes, snap-frozen in liquid nitrogen and stored at −70° C. Meanwhile, the 2 mL suspended bacteria remaining in the tube were snap-frozen in 0.5 mL aliquots in 3.5 mL clear lyophilization vials with a loose stopper.

Following 50 minutes incubation, one milliliter of the bacteria in the tube containing 2.5 mL or 5 mL of re-suspended bacteria was transferred to a fresh test tube and incubated on ice for 15 minutes in the presence of 35 μl of DMSO. After 15 minutes, an additional 35 μl of DMSO was added and 100 μl aliquots of cells were dispensed into 1.5 mL microfuge tubes, snap-frozen in liquid nitrogen and stored at −70° C. Meanwhile, 0.5 mL aliquots of the bacteria remaining in the tube were snap-frozen in 3.5 mL clear lyophilization vials with loose stoppers.

The remaining 2 tubes were centrifuged for 10 minutes at 1,500 g in a pre-cooled, fixed angle bucket centrifuge. 2.5 mL of CB-I was added to each one of the tubes and the tubes were re-suspended in the same way the first re-suspension was performed (with (+) or without (−) glass beads). The suspended bacteria in the tube containing glass beads were transferred to a fresh test tube. One milliliter from each tube was transferred to a fresh test tube and incubated on ice for 15 minutes in the presence of 35 μl DMSO. At the end of the incubation, 35 μl of DMSO was added to the tubes and gently mixed. 100 μl Aliquots of cells were dispensed into 1.5 mL microfuge tubes, snap-frozen in liquid nitrogen, and stored at −70° C.

Meanwhile, the suspended cells with no DMSO were snap-frozen in 0.5 mL aliquots in 3.5 mL clear lyophilization vials with loose stoppers. The frozen cells were stored at −70° C. for approximately 110 hours and then transferred to a pre-chilled lyophilizer.

A control treatment was performed in parallel according to the protocol of Example 3, above, using CB-I containing sucrose.

The lyophilization program outlined above in Example 1, Table 1, was used in the lyophilization process.

One vial of each experiment was taken for transformation. The rest of the vials were stored at −20° C. On the following day some of the stored vials were used for a second transformation.

Transformation

The “control” bacteria which were not lyophilized were defrosted on ice for 10 minutes while vials containing the lyophilized bacteria were re-suspended in Re-hydration Buffer (500 μl/vial) and incubated on ice for the same period of time (10 minutes). Following the incubation, the re-hydrated lyophilized bacteria were transferred to microfuge tubes in 100 μl aliquots.

Ten nanograms of PUC19 plasmid was added to the control and lyophilized bacteria samples (in the microfuge tubes), mixed by a gentle finger tapping on the tubes, and incubated on ice for 30 minutes. The cells were then subjected to a heat shock. The tubes were transferred to a 42° C. water bath for 30 seconds and then transferred back to the ice bucket for 2 minutes. 900 μl of SOC medium was added to each one of the test tubes and samples were shaken for 1 hour at 37° C.

The transformed bacteria in each one of the tubes was diluted 1:20 and 1:100 (for the lyophilized cells) or 1:200 and 1:2000 (for the non-lyophilized cells). Fifty microliters of the transformed bacteria from each one of the dilutions were plated on a LB-AMP plate and grown at 37° C. Colonies were counted after approximately 16 hours.

TABLE 7
Example 5 Transformation results

| | Yield before | Yield after |
|---|---|---|
| | lyophilization | lyophilization |
| Treatment | Colonies/μg plasmid | Colonies/μg plasmid |
| Lyophilized bacteria, − | 2.7x 10E8 | 1.5x 10E5 |
| glass beads | 2.2x 10E8 | approx. 1x 10E6 |
| | 2.2x 10E8 | |
| Lyophilized bacteria + | 2.4x 10E8 | 1.5x 10E6 |
| glass beads | 2.4x 10E8 | approx. 1x 10E6 |
| | 4x 10E8 | |
| | 2.2x 10E8 | |
| Lyophilized bacteria, | 2.7x10E8 | 3.2x 10E6 |
| dispensing after first | 3x 10E8 | 1.8x 10E6 |
| centrifugation | 3.2x 10E8 | |
| | 3.1x 10E8 | |
| Lyophilized bacteria re- | 1.5x 10E8 | approx. 1x 10E6 |
| suspended in 5 mL CB-I, | 2.5x 10 E8 | 6x 10E6 |
| dispensing after 50 | 2.3x 10E8 | |
| minutes on ice, no second | | |
| centrifugation. | | |
| Lyophilized bacteria, | approx. 1x 10E8 | 5x 10E5 |
| re-suspended in 2.5 mL | 1.6x 10E8 | approx. 1x 10E5 |
| CB-I, dispensing after 50 | 1.8x 10E8 | |
| minutes on ice, no second | | |
| centrifugation. | | |
| Lyophilized bacteria, | 1.5x 10E8 | 1.3x 10E6 |
| Control, three | 2.2x 10E8 | 5x 10E5 |
| centrifugations | 2x 10E8 | |

As various changes could be made in the above examples without departing from the scope of the invention, it is intended that all matter contained in the above examples be interpreted as illustrative and not in a limiting sense.

What is claimed is:

1. A process for producing lyophilized competent cells comprising:
(a) rendering eukaryotic or prokaryotic cells competent; and
(b) lyophilizing the competent cells.

2. The process of claim 1 wherein the eukaryotic or prokaryotic cells are grown in a medium within the temperature range of 28° C. to 40° C. prior to being rendered competent.

3. The process of claim 1 wherein the competent cells are snap-frozen prior to being lyophilized.

4. The process of claim 2 wherein the temperature of the growth conducive medium is at least 30° C.

5. The process of claim 2 wherein the temperature of the growth conducive medium is at least 37° C.

6. The process of claim 1 wherein the competent cells are snap-frozen and stored between about −80° C. and about −70° C. prior to lyophilization.

7. The process of claim 6 wherein the frozen competent cells are stored for at least one month prior to lyophilization.

8. The process of claim 6 wherein the frozen competent cells are stored for up to 2 months prior to lyophilization.

9. The process of claim 1 wherein lyophilizing the competent cells comprises lyophilizing the frozen cells at a starting temperature of about −30° C. or lower that gradually increases to about 25° C.

10. The process of claim 9 wherein the frozen competent cells are lyophilized over a period of between about 20 and about 30 hours.

11. The process of claim 1 wherein the lyophilized competent cells are shipped to a remote location at a temperature within the range of about 0° C. to about 25° C.

12. The process of claim 1 wherein the lyophilized competent cells are shipped to a remote location at a temperature within the range of about 0° C. to about 8°.

13. The process of claim 1 wherein the lyophilized competent cells are shipped to a remote location at a temperature within the range of about 0° C. to about 4° C.

14. The process of claim 1 wherein the lyophilized competent cells are stored at a temperature within the range of about −70° C. to about 8° C.

15. The process of claim 1 wherein the lyophilized competent cells are stored at a temperature within the range of about −20° C. to about 25° C.

16. The process of claim 1 wherein the lyophilized competent cells are stored at a temperature within the range of about −20° C. to about 8° C.

17. The process of claim 1 wherein the prokaryotic cells are gram negative bacteria.

18. The process of claim 17 wherein the bacteria are of a genus selected from the group consisting of Escherichia, Agrobacterium, Klebsiella, Proteus, Pseudomonas, Rhizobium, Salmonella, and Shigella.

19. The process of claim 18 wherein the cells are E. coli.

20. The process of claim 19 wherein the E. coli is selected from the group of strains consisting of RR1, HB101, JM101, JM109, DH5α, DH1, LE392, and BL21.

21. The process of claim 1 wherein the lyophilized competent cells are re-hydrated in a solution containing dimethyl sulfoxide.

22. The process of claim 21 wherein the solution further contains mercaptoethanol.

23. The process of claim 1 wherein the eukaryotic or prokaryotic cells are rendered competent in a buffer containing a stabilizer.

24. The process of claim 23 wherein the stabilizer is selected from the group consisting of sucrose, trehalose, TB-Z, galactose, glucose, maltose, raffinose, lactose, inositol, ectoine, and proline.

25. The process of claim 24 wherein the stabilizer is sucrose or trehalose.

26. The lyophilized competent cells according to claim 1 wherein the cells have a transformation efficiency of at least 5×10⁵ transformations per microgram of DNA.

27. Lyophilized competent cells produced by the process of claim 1.

28. A process for producing lyophilized competent cells comprising:
(a) growing cells in a medium at a temperature of about 37° C.;
(b) rendering the grown cells competent in a solution containing sucrose, trehalose, or mixture thereof;
(c) snap-freezing the competent cells; and
(d) lyophilizing the snap-frozen competent cells.

29. The process of claim 28 wherein the lyophilized competent cells are re-hydrated and induced to take up exogenous DNA.

30. The process of claim 29 wherein the lyophilized competent cells are re-hydrated in a solution containing dimethyl sulfoxide.

31. The process of claim 29 wherein the lyophilized competent cells are shipped to a remote location at a temperature within the range of about 0° C. to about 25° C.

32. The process of claim 28 wherein the lyophilized competent cells are shipped at a temperature within the range of about 0° C. to about 8° C.

33. The process of claim 28 wherein the lyophilized competent cells are shipped at a temperature within the range of about 0° C. to about 4° C.

34. The process of claim 28 wherein the lyophilized competent cells are stored at a temperature within the range of −20° C. to 25° C.

35. The process of claim 28 wherein the lyophilized competent cells are stored at a temperature within the range of −20° C. to 8° C.

36. Lyophilized competent cells produced by the process of claim 28.

37. A process for produc ing lyophilized competent cells comprising lyophilizing competent cells.

38. The lyophilized competent cells of claim 37 wherein the cells are of a genus selected from the group consisting of Escherichia, Agrobacterium, Klebsiella, Proteus, Pseudomonas, Rhizobium, Salmonella, and Shigella.

39. The lyophilized competent cells of claim 38 wherein the cells are E. coli.

40. The lyophilized competent cells of claim 39 wherein the E. coli is selected from the group of strains consisting of RR1, HB101, JM101, JM109, DH5α, DH1, LE392, and BL21.

41. The lyophilized competent cells of claim 37 wherein the cells are of a genus selected from the group consisting of Escherichia, Agrobacterium, Klebsiella, Proteus, Pseudomonas, Rhizobium, Salmonella, and Shigella.

42. The lyophilized competent cells of claim 41 wherein the cells are E. coli.

43. The lyophilized competent cells of claim 42 wherein the E. coli is selected from the group of strains consisting of RR1, HB101, JM101, JM109, DH5α, DH1, LE392, and BL21.

44. Lyophilized competent cells.
